(19) United States
(12) Patent Application Publication
(10) Pub. No.: US 2021/0205183 A1
(43) Pub. Date: Jul. 8, 2021

(54) Makeup Cosmetic Having Improved Impregnated Material

(71) Applicant: LG Household & Health Care Ltd., Seoul (KR)

(72) Inventors: Sung-Soo Kang, Daejeon (KR); Hyeon-Jeong Kim, Daejeon (KR); Sang-Wook Park, Daejeon (KR)

(73) Assignee: LG Household & Health Care Ltd., Seoul (KR)

(21) Appl. No.: 17/208,405

(22) Filed: Mar. 22, 2021

Related U.S. Application Data
(63) Continuation of application No. 15/773,758, filed on May 4, 2018.

(51) Int. Cl. A61K 8/02; A61K 8/87; A61Q 19/00; A61K 8/06; A61Q 1/02; A61Q 17/04

(52) U.S. Cl. CPC A61K 8/0204; A61K 8/87; A61Q 19/00; A61K 2800/87; A61Q 1/02; A61Q 17/04; A61K 8/064

(57) ABSTRACT

The present invention relates to a method for preparing a cosmetic product having a porous impregnated material impregnated with a cosmetic composition; and an applicator, wherein the porous impregnated material has a compression part on one surface contacted by the applicator so that the cosmetic product is capable of having content dispensed uniformly from the outset, and can prevent an excessive amount of content from being dispensed at the outset.

### CROSS-REFERENCE TO RELATED APPLICATION

This application is a continuation of U.S. application Ser. No. 15/773,758, filed May 4, 2018, which is a national phase entry under 35 U.S.C. § 371 of International Application No. PCT/KR2015/011930, filed Nov. 6, 2015, the disclosures of which are incorporated herein by reference.

### TECHNICAL FIELD

The present disclosure relates to a makeup cosmetic product including an improved impregnation material in which a cosmetic agent is supported. More particularly, the present disclosure relates to a makeup cosmetic product capable of controlling the discharge amount of the impregnated cosmetic agent consistently.

### BACKGROUND ART

In general, water-in-oil (W/O) type cosmetic agent or oil-in-water (O/W) type cosmetic agent having fluidity is contained in a tube or a pump-like formulation for use. However, there is a problem in that such a formulation is relatively inconvenient to carry and use.

According to the related art, JP 2003-12457, KR 10-1159877 and EP 0 528 705 disclose an easily portable product including a cosmetic agent supported in a porous impregnation material, such as urethane foam, and applied to the skin by using an applicator, such as a puff.

The sponge for impregnation suggested by the above-mentioned related art is obtained through a foaming process. The cells formed in the sponge are mostly open cells and have a porous structure capable of supporting a cosmetic agent.

The internal space formed during the foaming process of the sponge has a large pore size to allow easy impregnation. However, there is a problem in that the content is discharged excessively when the impregnation material is pressurized by a puff for application. Particularly, when the impregnation material is pressurized by the hand without using a separate puff for application, the user can sense discharge of the content through the sense of the hand. However, when the content is dispensed by a separate puff and used, discharge of the content is sensed less, and thus the content is discharged excessively at the initial time of use.

When the content is discharged excessively from the impregnation material, a large amount of cosmetic agent is used at the initial time undesirably to cause degradation of cost-efficiency. In addition, it is difficult to apply the cosmetic uniformly and finely to the face, thereby making it difficult to realize proper skin makeup.

To solve the above-mentioned problems, many studies have been conducted continuously to control the discharge amount of a cosmetic agent impregnated in a sponge adequately.

Korean publication No. 2013-0116043 discloses a cosmetic container using sponges having different physical properties. This is intended to facilitate discharge of an impregnated cosmetic composition through a sponge having a larger number of pores. However, there is no disclosure about the problem to be solved by the present disclosure, i.e., maintaining the discharge amount of a cosmetic composition consistently.

According to the related art, there has been a study about controlling the amount of a cosmetic agent discharged from an impregnation material by adding a structure, such as a screen net, onto the impregnation material. However, after applying the structure actually to a product, there were problems in that no consistent discharge amount was observed and significantly high cost was required to fabricate the cosmetic product.

Therefore, the inventors of the present disclosure have conducted studies to develop a cosmetic product which provides a consistent amount of cosmetic agent discharged from an impregnation material and is convenient to use. The present disclosure is based on this study.

### DISCLOSURE

### Technical Problem

The present disclosure is designed to solve the problems of the related art, and therefore the present disclosure is directed to providing an impregnation material capable of maintaining the amount of a cosmetic composition discharged therefrom consistently, and a cosmetic product including the same.

### Technical Solution

To solve the above-mentioned problem, there is provided a cosmetic product which is convenient to use and provides a consistent discharge amount of cosmetic composition.

In one aspect of the present disclosure, there is provided a cosmetic product including a porous impregnation material in which a cosmetic composition is supported; and a separate applicator with which the cosmetic composition is dispensed from the porous impregnation material and is applied to the skin, wherein the impregnation material includes a compressed part on one surface thereof to be in contact with the applicator.

After the inventors of the present disclosure conducted many studies for a long time, we have recognized that a porous impregnation material having an open cell structure is convenient to support and use a cosmetic composition but shows an excessive initial discharge amount, thereby making it difficult to control the discharge amount when using an applicator. The inventors of the present disclosure completed the present disclosure based on this.

Although some methods for controlling a discharge amount have been suggested, there is no method for controlling a discharge amount consistently, effectively from the initial time of use.

The term ‘impregnation’ means filling a space by dipping it in a fluidic ingredient, such as a liquid. As used herein, ‘impregnation’ refers to a porous impregnation material impregnated with a cosmetic composition and is used in its broad meaning to cover the porous impregnation material to which the cosmetic composition is absorbed.

As used herein, ‘impregnation material’ may include any material in which a cosmetic composition can be impregnated and may have a porous form, such as a sponge. With a view to the present disclosure, the impregnation material preferably has an open cell structure, more preferably is porous foam including pores having an open cell structure and formed during a foaming process, and most preferably is foamed urethane.

The applicator means an instrument with which the cosmetic composition is dispensed and applied to the face. There is no particular limitation in the material or shape of the applicator, as long as the applicator can dispense the fluidic cosmetic composition and apply it to the skin.

According to the present disclosure, one surface of the impregnation material to be in contact with the applicator has a compressed part to control the discharge amount of the cosmetic agent. In other words, the inventors of the present disclosure have experimentally found that a pore size decreased relatively as compared to an uncompressed part allows the discharged cosmetic agent to be retained in the compressed part for a longer time, and thus can prevent excessive discharge of the cosmetic composition at the initial time.

Particularly, an applicator is mounted to a press machine (e.g. LLOYD LS1) capable of applying the same force, a pressure of 0.4 kgf/cm² is applied to the impregnation material in which a cosmetic composition is supported according to the present disclosure for 3 seconds, and then the amount of cosmetic composition dispensed with the applicator is determined so that the discharge amount may be compared as a function of the number of times the pressure is applied.

The discharge amount of cosmetic composition dispensed with an applicator from the impregnation material having a compressed part according to the present disclosure may show a variation less than 0.03 g, preferably less than 0.02 g and more preferably less than 0.015 g from the initial pressurization time to 10 times of pressurization.

The compressed part may be obtained by various compression methods capable of inducing compression of the impregnation material, preferably by using thermal compression.

The thermal compression method used herein may be interchanged with thermal pressurization and means a compression method inducing an instant decrease in volume under the application of high temperature. The term, thermal compression, is used herein in its broad meaning to cover not only a thermal compression method for intermetallic bonding but also a method for decreasing volume under the application of high temperature and pressure.

Particularly, the thermal compression method includes pressurizing the impregnation material with a heated metallic plate and may use a pressurization system having such a structure that heated oil may be circulated in the system to transfer heat at 500° C. or higher. Preferably, heat may be transferred at around 650° C. The pressurization system for use in manufacturing the impregnation material having a compressed part may include a cylindrical pressurization roll or heated press machine. According to the present disclosure, the thermal compression method for a sponge may be carried out in the conventional manner and is not particularly limited. The surface of the pressurization roll or press machine may have a predetermined pattern by forming protrusions or grooves. This may allow carving of various patterns on the top surface of the compressed part.

The compressed part of the impregnation material may be formed to provide a ratio of [length of compressed part:total length of impregnation material] of 1:4-1:6, preferably 1:4.5-1:5.5.

For example, a foam having a cell number of 45 ppi-65 ppi or a foam having a density of 0.01-0.03 g/cm³ may be thermally compressed in the direction of contact with an applicator so that the height of the foam may be reduced by 10-30%, preferably 12-28%, and more preferably 15-25% as compared to the height before the thermal compression.

When the ratio of the compressed part is increased more, the cost required for manufacturing the impregnation material is increased and the content discharge ability may be lowered. When the ratio of the compressed part is decreased, it may be not possible to obtain the desired effect of the present disclosure sufficiently and it may be difficult to carry out uniform compression and shaping.

The compressed part may have a density corresponding to 4-6 times of the density of the uncompressed part of the impregnation material.

The cosmetic product according to the present disclosure is obtained by impregnating the impregnation material having a compressed part with a cosmetic composition. The cosmetic composition is a fluidic cosmetic composition and may have fluidity under 1 atm at 25° C.

Preferably, the cosmetic composition may be water-in-oil (W/O) type cosmetic composition and preferably includes foundation, sunscreen, or the like. The ingredients contained in the foundation or sunscreen may be those used currently in the art.

The cosmetic product according to the present disclosure may further include other elements used for conventional cosmetic products, in addition to the compressed impregnation material and applicator.

For example, in the case of a compact container-type cosmetic product, it may further include a mirror and a casing capable of separating the impregnation material from the applicator. However, the present disclosure is not limited thereto and any elements used currently for conventional cosmetic products may be used.

In another aspect of the present disclosure, there is also provided a urethane foam impregnation material for a cosmetic composition which has a compressed part formed on one surface thereof.

The urethane foam impregnation material may have a porous open cell structure and the compressed part may have a density corresponding to 4-6 times of the density of the uncompressed part.

### Advantageous Effects

The cosmetic product according to the present disclosure allows consistent discharge of the content from the initial time.

It is possible to solve the problem of excessive discharge of the content from the impregnation material and a gradual decrease in discharge amount, and to maintain a constant discharge amount.

It is possible to apply the cosmetic according to the present disclosure finely and uniformly to the face.

### DESCRIPTION OF DRAWINGS

FIG. 1 shows the cell structure of an uncompressed impregnation material and that of a compressed impregnation material, wherein the cell diameter of the compressed impregnation material is approximately 1.44 times smaller than that of the uncompressed impregnation material to form a dense cell structure.

FIG. 2 illustrates various patterns that may be carved on an impregnation material.

### BEST MODE

Hereinafter, the present disclosure will be described in detail with reference to the following examples. However, it should be understood that the following examples are not intended to limit the scope of the present disclosure and various changes and modifications may be made without departing from the spirit and scope of the invention as defined in the following claims. It will be apparent that these exemplary embodiments are provided so that the present disclosure will be complete and understood easily by those skilled in the art.

### Example 1. Manufacture of Impregnation Material Having Compressed Part

A cylindrical polyurethane impregnation sponge having a diameter of 48 mm and a length of 11 mm was used. The sponge (urethane foam) includes a compressed part up to 2 mm from the top and an uncompressed part not subjected to thermal compression. Herein, EZ88DH available from Foam Tec was used.

### Example 2. Determination of Physical Properties of Compressed Impregnation Material

1. Density

The difference in density between an impregnation material compressed to 2 mm from the top surface and an uncompressed conventional impregnation material was determined.

As can be seen from the following Table 1, the impregnation material including a compressed part has higher density as a whole.

TABLE 1
Density (g/cm³)

| | Conventional sponge | Compressed sponge |
|---|---|---|
| 1 | 0.022 | 0.031 |
| 2 | 0.022 | 0.030 |
| 3 | 0.021 | 0.031 |
| 4 | 0.021 | 0.031 |
| 5 | 0.021 | 0.031 |
| 6 | 0.022 | 0.031 |
| 7 | 0.021 | 0.031 |
| 8 | 0.022 | 0.031 |
| 9 | 0.021 | 0.032 |
| 10 | 0.021 | 0.031 |
| Average | 0.021 | 0.031 |

Meanwhile, after comparing the density of the 2 mm compressed part in the impregnation material having a compressed part with that of the 2 mm part in the uncompressed impregnation material, it can be seen that the compressed part has a density (0.106 g/cm³) approximately 5 times higher than the density (0.021 g/cm³) of the uncompressed part.

2. Cell Diameter

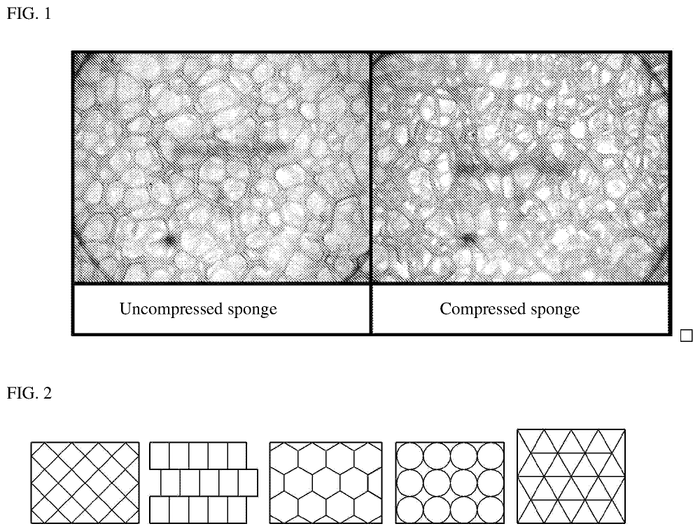

As can be seen from FIG. 1, the compressed impregnation material has a cell diameter approximately 1.44 times smaller than the cell diameter of the conventional impregnation material and forms a dense structure.

3. Cell Number

After determining the cell number of the uncompressed part and that of the compressed part, the compressed part has an average cell number of 79.9 ppi which is larger than the cell number (55.5 ppi) of the uncompressed part, as can be seen from the following Table 2.

TABLE 2

| | Uncompressed part | Compressed part |
|---|---|---|
| Cell number | 55.5 ppi | 79.9 ppi |

As can be seen from FIG. 1 and Table 2, the compressed part has a larger cell number and a relatively smaller pore size as compared to the uncompressed part. Thus, it is thought that the cosmetic agent supported in the impregnation material having a compressed part is not discharged excessively and a constant discharge amount can be maintained.

### Example 3. Preparation of Cosmetic Composition

The following composition and ratio were used to prepare a cosmetic composition.

TABLE 3

| Ingredients | Material (wt %) | Example |
|---|---|---|
| Oil phase | Cyclopentasiloxane | 17.0 |
| ingredients | Phenyl trimethicone | 14.0 |
| | Caprylic/capric triglyceride | 2.0 |
| | Ethylhexylmethoxy cinnamate | 7.5 |
| | PEG-10 dimethicone | 3.0 |
| | Sorbitane sesquioleate | 1.0 |
| | Methyl paraben | 0.1 |
| Thickener | Disteardimonium hectorite | 0.2 |
| Pigments | Titanium dioxide | 15.0 |
| | Yellow iron oxide | 0.9 |
| | Red iron oxide | 0.2 |
| | Black iron oxide | 0.1 |
| Aqueous phase | Purified water | To 100 |
| | Dipropylene glycol | 5.0 |
| | Salt | 1.0 |
| | Tromethamine | 2.5 |
| | Phenylbenzimidazole sulfonic acid | 4.0 |

### Example 4. Test for Determination of Discharge Amount

The impregnation material (Example 1) subjected to compression/pressurization and the non-treated impregnation material (Comparative Example) were used to compare the discharge amount at the initial stage (1-5 times) with the discharge amount after 6-10 times of use.

The test was carried out as follows.

A puff for application (available from S&P WORLD, cut into 1 cm×1 cm) was mounted to a press machine (LLOYD LS1) capable of pressurizing a sample to the same depth over the same area under the same force. Then, the amount of a cosmetic composition dispensed from the porous foam was calculated, when the porous foam (cut into a size of diameter 4.6 cm×height 1 cm) impregnated with the cosmetic composition was pressurized for 3 seconds under the same pressure (0.4 kgf/cm²). The unit is gram (g).

TABLE 4

| | Discharge number | | | | | | | | | |
|---|---|---|---|---|---|---|---|---|---|---|
| | 1 | 2 | 3 | 4 | 5 | 6 | 7 | 8 | 9 | 10 |
| Conventional sponge | 0.31 | 0.26 | 0.22 | 0.18 | 0.2 | 0.19 | 0.18 | 0.16 | 0.16 | 0.17 |
| Compressed sponge | 0.18 | 0.19 | 0.18 | 0.18 | 0.17 | 0.18 | 0.17 | 0.16 | 0.17 | 0.17 |

As shown in Table 4, after determining the pattern of discharge amount during use, it can be seen that the conventional impregnation material causes a rapid decrease in discharge amount, while the compressed impregnation material discharges the content consistently from the initial time.

### Example 5. User Test

The cosmetic product including the compressed impregnation material impregnated with the cosmetic composition was evaluated by twenty females in their 20s-30s based on a five-point scale of satisfaction levels, and the results are shown in the following Table 5 in terms of an average value. The evaluation is based on the easiness of discharge of the cosmetic composition.

TABLE 5

| | | Conventional | |
|---|---|---|---|
| | | sponge | Compressed sponge |
| Easiness of | First week | 3 | 5 |
| discharge | Second week | 5 | 5 |
| | Third week | 4 | 4 |

Most of the users gave a lower point to the conventional impregnation material for the first week, because the initial discharge amount was excessively large and the discharge amount was decreased rapidly. Then, they gave a lower point to both the conventional impregnation material and the compressed impregnation material from the third week, since both impregnation materials provided a decreased discharge amount and were less satisfied.

### INDUSTRIAL APPLICABILITY

According to the present disclosure, it is possible to provide an impregnation material capable of maintaining a constant amount of cosmetic composition discharged therefrom, and a cosmetic product including the same.

What is claimed is:

1. A manufacturing method for preparing a cosmetic product comprising an applicator and a porous impregnation material discharging an impregnated water-in-oil emulsion cosmetic composition when the applicator presses a top surface of the porous impregnation material,
wherein the manufacturing method comprises:
preparing a porous material which is a single urethane foam having an open cell structure;
compressing the top surface of the single urethane foam with a heated plate at a temperate of 500° C. or higher so that a height of the urethane foam is reduced, forming a compressed part and an uncompressed part;
impregnating the cosmetic composition in the single urethane foam to be supported therein, and
providing an applicator with which the cosmetic composition is dispensed from the porous impregnation material,
wherein (a) the compressed part is formed on the top surface via thermal compression by pressurizing the top surface of the porous impregnation material to be in contact with the applicator, and (b) the uncompressed part is provided under the compressed part which the thermal compression is not applied, and
wherein the top surface of the compressed part is configured to discharge the cosmetic composition when the applicator pressurizes the top surface of the compressed part of the porous impregnation material.

2. The manufacturing method of claim 1, wherein a ratio of a length of the compressed part:a length of the single urethane foam is 1:4-1:6.

3. The manufacturing method of claim 1, wherein a density of the compressed part is 4 to 6 times greater than a density of the uncompressed part.

4. The manufacturing method of claim 1, wherein the single urethane foam has a cell number of 45 ppi (pore per inch) to 65 ppi before the thermal compression.

5. The manufacturing method of claim 4, wherein the single urethane foam has a density of 0.01 to 0.03 g/cm3 before the thermal compression.

6. The manufacturing method of claim 5, wherein a height of the single urethane foam is reduced by 10% to 30% after the thermal compression when compared to a height before thermal compression.

7. The manufacturing method of claim 1, wherein the top surface of the compressed part has patterns.

8. The manufacturing method of claim 1, wherein the cosmetic composition comprises a pigment, wherein the pigment comprises titanium dioxide or iron oxide.

9. The manufacturing method of claim 1, wherein when an amount of the cosmetic composition dispensed with the applicator upon pressurization of the compressed part under a pressure of 0.4 kgf/cm² for 3 seconds is measured 10 times, the amount varies less than 0.03 g per each time.
